(19) United States
(12) Patent Application Publication
(10) Pub. No.: US 2014/0024961 A1
(43) Pub. Date: Jan. 23, 2014

(54) SYSTEM AND METHOD FOR DETECTING HUMAN EMOTION

(71) Applicant: NATIONAL TAIWAN UNIVERSITY, Taipei City (TW)

(72) Inventors: Keng-Sheng LIN, Taipei City (TW); Chen-Gia TSAI, Taipei City (TW); Homer H. CHEN, Taipei (TW)

(73) Assignee: National Taiwan University, Taipei City (TW)

(21) Appl. No.: 13/777,450

(22) Filed: Feb. 26, 2013

(30) Foreign Application Priority Data
Jul. 18, 2012 (TW) 101125776

(51) Int. Cl. A61B 5/16; A61B 5/00; A61B 5/053

(52) U.S. Cl. CPC A61B 5/165; A61B 5/0531; A61B 5/7278; A61B 5/7405; A61B 5/7228; A61B 5/6898
USPC 600/547

(57) ABSTRACT

An emotion excitement detection system includes a detection unit for engagement with human skin, an amplifier unit connected to the detection unit to amplify signal detected by the detection unit, a first control unit connected to the amplifier unit for measurement of skin conductance after the detection unit engages with the human skin and transmit result of the measurement and a processing unit producing a skin conductance slope in response to the skin conductance and excitement degree resulted from the skin conductance slope.

### CROSS-REFERENCE TO RELATED APPLICATION

This application claims priority of application No. 101125776, filed on Jul. 18, 2012 in the Intellectual Property Office of The Republic of China.

### FIELD OF THE INVENTION

The invention relates to a human emotion detection system which is composed of a detection unit, an amplifier unit, a first control unit and a processing unit and, more particularly, to human emotion detection method having the steps of measuring human skin conductance, amplifying the skin conductance, and calculating a skin conductance slope based on the skin conductance.

### BACKGROUND OF THE INVENTION

Emotion is an affective state of consciousness in which joy, sorrow, fear, hate, or the like, is experienced. Human emotion may undergo subtle or swift changes due to exterior stimulus displayed in the form of, for example, music or video. In recent years, there is an increasing interest in the design of systems and devices that can detect and interpret human emotions. Skin conductance is one of the most commonly used methods for interpreting human emotion. A skin conductance response (SCR) is characterized by an abrupt increase of skin conductance, which is used as an indication of psychological or physiological arousal. The principle is that, when a person is in the hyper-emotional status, the autonomic sympathetic nerve system will automatically kick in to increase sweat, which, in turn, raises the skin conductance. On the contrary, when the person at test is in calm, the skin conductance is reduced. Researchers have found that skin conductance is highly related to emotion fluctuations.

The relevance can be illustrated by a real example. FIG. 1 shows the sound wave of a pop song and the corresponding time course of the SCR induced by the song. The SCR, which is averaged across 39 listeners, shows the listeners' feeling fluctuation over the observation time period, and the peaks/anti-peaks represent the moments of extreme excitement/relax. Based on these peaks/anti-peaks, specific musical events that demonstrate emotion-evoking power can be identified. The fact that the SCR curve drops below the baseline at (a) indicates that the listeners feel satisfied emotionally. Next, the coming of verse causes a peak in the SCR curve at (b) as listeners change their focuses to the vocal voice. The subsequent sentimental section causes an obvious drop in the SCR curve at (c). Then, the twist from the verse to chorus causes another peak at (d). Finally, when the song is over, the SCR curve drops below baseline at (e) as the listeners feel satisfied.

There are skin conductance measurement devices available in the market, such as the Q Sensor made by Affectiva. However, such devices suffer from the following two drawbacks. First, these devices use skin conductance level as an emotion index. But the skin conductance level is not an effective measure of subtle and swift emotional changes. Besides, the skin conductance level depends not only on the psychological status of the individual, but also on physiological and environmental factors such as the moisture level of the skin, blood flow, ambient temperature, and the level of humidity. As a result, the SCR shows significant inter-subject and intra-subject differences. Second, these devices are connected to computers through certain auxiliary equipment for special interface, making the system cumbersome and difficulty to use.

This invention discloses an SCR measurement method for the human emotion detection to resolve the aforementioned problems of the prior art. It can be implemented on a smart phone so that the user can easily detect or record his/her emotional response while using the smart phone.

### SUMMARY OF THE INVENTION

The primary objective of the present invention is to provide an emotion detection system which has high temporal resolution and is portable as well as readily for connection with other devices.

In order to achieve the aforementioned objective, the system of the present disclosure comprises:

a detection unit for engagement with human skin;

an amplifier unit connected to the detection unit to amplify signal detected by the detection unit;

a first control unit connected to the amplifier unit for measurement of skin conductance after the detection unit engages with the human skin and transmit result of the measurement; and

a processing unit computing a skin conductance slope in response to the skin conductance and excitement degree resulted from the skin conductance slope.

In a preferred embodiment of the present disclosure, the first control unit modulates the skin conductance into an audio signal for transmission.

In a preferred embodiment of the present disclosure, the system of the present invention further has a mobile electronic device to display the excitement degree.

In a preferred embodiment of the present disclosure, the mobile electronic device has a second control unit and the processing unit, the second control unit is to receive the audio signal and demodulate the audio signal into a skin conductance signal, the processing unit produces the skin conductance slope in response to the skin conductance signal.

In still a preferred embodiment of the present disclosure, the mobile electronic device further includes a wireless transceiver module for transmitting the audio signal.

Still, in a preferred embodiment of the present invention, the system of the present invention further has a play unit to play multimedia files in response to the skin conductance slope.

It is another objective of the present invention to provide an emotion status detection method which comprises the steps of:

measuring skin conductance;

amplifying the skin conductance; and

computing a skin conductance slope based on the skin conductance.

In a preferred embodiment of the present invention, a step of modulating the skin conductance into an audio signal; and demodulating the audio signal into a skin conductance signal after the amplifying step.

In a preferred embodiment of the present invention, in the skin conductance transforming step, a pulse width modulation technique is adopted for the modulation.

In a preferred embodiment of the present invention, in the audio signal transforming step, the audio signal is processed to obtain the frequency of the audio signal and then the audio signal is demodulated to obtain the skin conductance signal.

In still a further preferred embodiment of the present invention, a step of transmitting the audio signal through a wireless transceiver module to a wireless device.

Still, in a preferred embodiment of the present invention, a step of playing a suitable multimedia file format in response to the skin conductance slope.

The fluctuations (about 0.01 μS) of skin conductance response (roughly about 5-20 μS) is small. Technologies used in the conventional measures normally neglect such phenomenon and just simply amplify what is observed without any in-depth analysis, resulting in poor performance.

In order to obviate the aforementioned problems, the present invention adopts Wheatstone bridge to monitor such small variations of skin conductance response. Specifically, the Wheatstone bridge, which is a circuit, is specifically designed to monitor changes of skin conductance response in micro level.

Furthermore, while conventional systems require expensive auxiliary device for connection with a general purpose computer for post process after the acquirement of the skin conductance response, the technique and system of the present invention adopt pulse width modulation (PWM) to modulate the skin conductance response into an audio signal for transmission. As a result, an audio socket is sufficient for transmission of the skin conductance signal. This is a practical solution because most smart phones are equipped with audio socket such that the user of the smart phone is able to check his/her emotional status at any time and any place.

Lastly, as musical incidents such as the start and finish of the verse and chorus can cause skin conductance level to change, smart-phone users when listening to music can record his/her own skin conductance changes in response to the emotion expressed by the music. As a result, the listeners can check his/her SCR after the musical incidents or to compare his/her SCR curve with others so as to understand the music tastes of self and of others. Plenty of fun is thereby added to music listening.

### BRIEF DESCRIPTION OF THE DRAWINGS

FIG. 1 shows the time course of SCR induced by a song, which was averaged across 39 listeners.

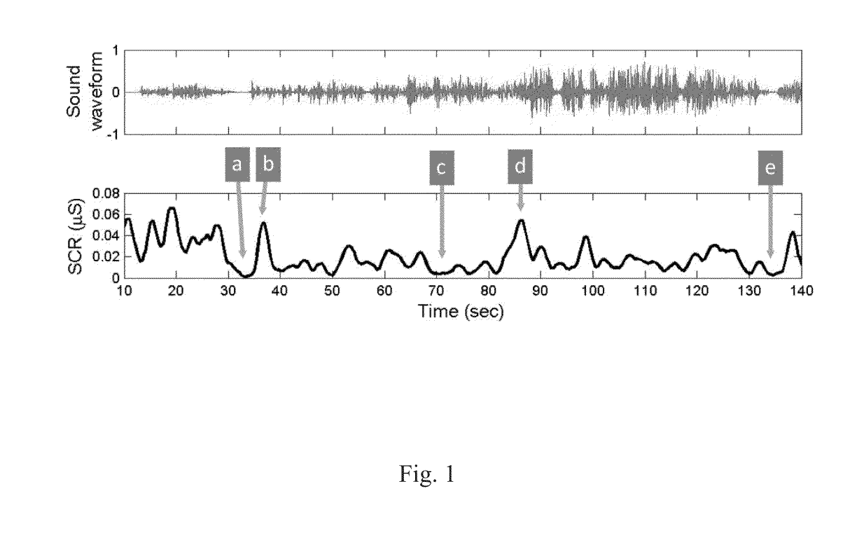

FIG. 2 is a schematic view showing the structure of the emotion detection system of the present invention.

FIG. 3 is a schematic view showing the electrical diagram of the emotion detection system of the present invention.

FIG. 4 is a flow chart showing steps implemented in the present invention.

### DETAILED DESCRIPTION OF THE INVENTION

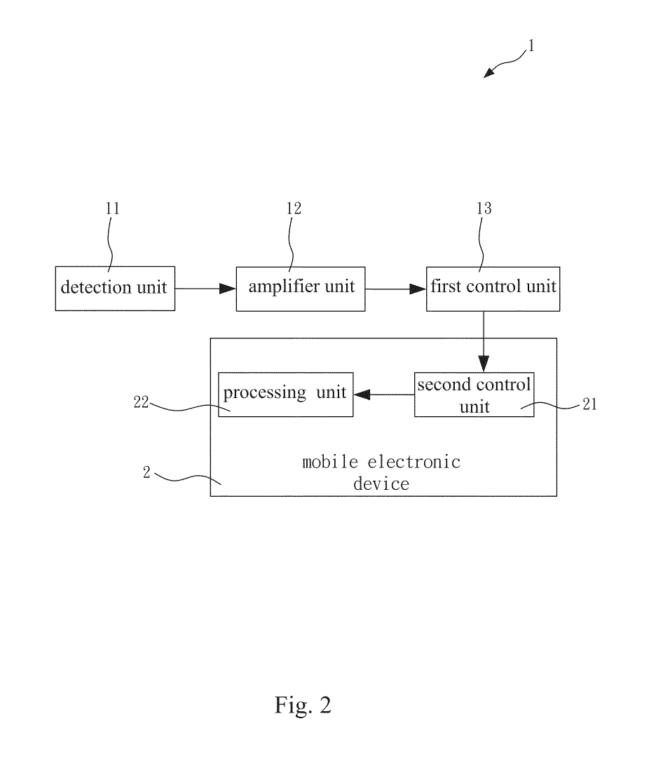

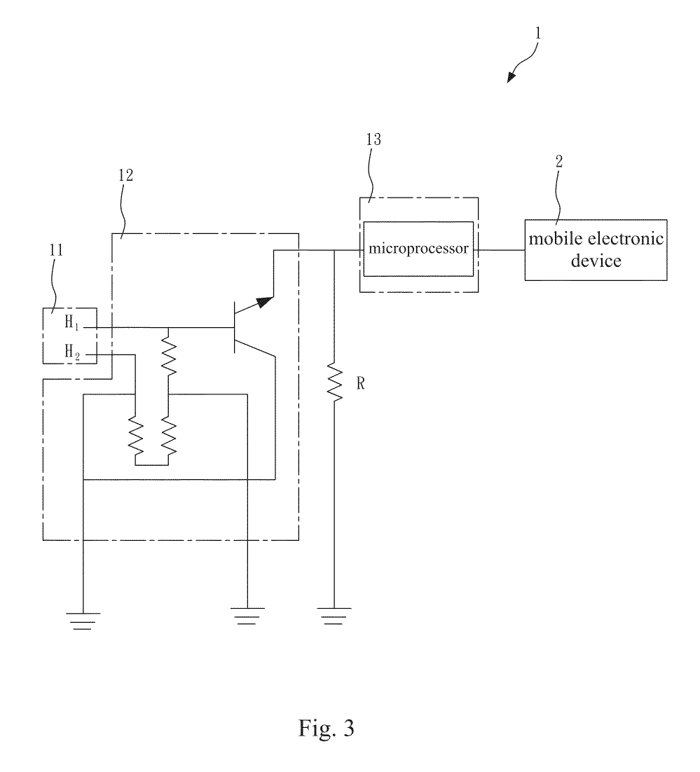

As shown in FIGS. 2 and 3, the human emotion detection system 1 constructed in accordance with the preferred embodiment of the present invention is connected to a mobile electronic device 2 and has a detection unit 11, an amplifier unit 12, a first control unit 13 and a processing unit 22.

The detection unit 11 is provided for engagement with human skin and with two prongs to engage with the human skin.

The amplifier unit 12 is connected to the detection unit 11 and adopts a Wheatstone bridge and a transistor.

The first control unit 13 is connected to the amplifier unit 12 so that when the detection unit 11 is in engagement with the human skin, the first control unit 13 is able to measure the skin conductance response (SCR) which is amplified by the amplifier unit 12. The first control unit 13 modulates the skin conductance response into an audio signal for transmission. In the preferred embodiment of the present invention, the modulating function of the first control unit 13 is accomplished by a microcontroller. Using microcontroller to modulate the skin conductance response into the audio signal is well known in the art and thus detailed description thereof is omitted for maintaining brevity of the description. Then the audio signal is transmitted to the mobile electronic device 2 via an audio socket. In a preferred embodiment of the present invention, the audio socket is a TRRS socket (four-contact earphone socket).

The mobile electronic device 2 includes a second control unit 21 and the processing unit 22. The second control unit 21 is used to pick up the audio signal and then modulate the audio signal into a skin conductance signal. The processing unit 22 is able to compute a skin conductance slope based on the skin conductance signal and estimate an excitement degree. The skin conductance slope is then processed to become the excitement degree. In the embodiment of the present invention, the mobile electronic device 2 can be a smart phone, a flat screen computer, a pad or the like. A part of the technology can be accomplished by programming, which is easily understood by persons skilled in the art.

In practice, the detection unit 11, the amplifier unit 12 and the first control unit 13 can be implemented all together in a wrist band for wearing on the arm or the like. With the assistance of an audio plug in the audio socket in the mobile electronic device 2, the user wearing the wrist band containing therein the detection unit 11, the amplifier unit 12 and the first control unit 13 can have the excitement degree anytime and have the final result displayed in the mobile electronic device 2.

Furthermore, the system of the present embodiment has a player device which is able to play multimedia files corresponding to the resulting skin conductance slope. The player device may be implemented inside the mobile electronic device 2 and is able to play the multimedia files via the speaker or the screen of the mobile electronic device 2. The multimedia files may be music files, films, or pictures each designated with a specific feature corresponding to a certain degree of excitement. For example, when the user is emotionally aroused, the player device may play soft music to achieve the purpose of calming down the user's emotion. As such, the present disclosure discloses an emotion-based song retrieval technology.

To be more specific, the mobile electronic device is only an option for the system of the present disclosure, meaning that it may be replaced with any equivalent electronic devices having the same or almost the same function as the mobile electronic device described earlier. Also, the mobile electronic device 2 is not required to modulate the skin conductance response into an audio signal for transmission. It is a preferred embodiment only because most currently available mobile communication devices are equipped with audio socket(s) or at least a wireless transceiver module. However, it should be apparent to those skilled in the art that the present invention may be practiced by transmitting the skin conductance response by other means such as Bluetooth or near field communication (NFC). In this embodiment the SCR data are modulated and sent to the wireless transceiver module which has built-in application protocols, such as Bluetooth or near field communication.

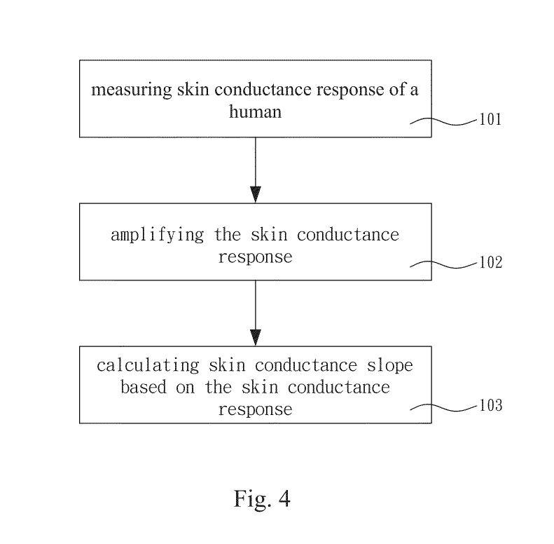

With reference to FIG. 4, it is to be noted that the emotion detection method of the preferred embodiment of the present disclosure includes the steps of:

101 measuring skin conductance response of a human;

102 amplifying the skin conductance response; and

103 calculating skin conductance slope based on the skin conductance response.

In steps 101 and 102, two prongs are provided for engagement with human skin for measurement of the human conductance response. In another possible application, a Wheatstone bridge is used to measure the human conductance response and a transistor is used to amplify the response to precisely measure the smallest change of the human conductance response. Furthermore, the resistance R in FIG. 3 is used to transform a current signal which is amplified by the transistor into a voltage signal.

After step 102, the preferred embodiment of the present disclosure further has a step of modulating the human skin conductance signal into an audio signal 1021 and a step of demodulating the audio signal into a human skin conductance signal 1022.

In step 1021, the PWM technology of the microcontroller is used to modulate the human skin conductance into the audio signal. And after the audio signal is successfully modulated, the audio signal is then transmitted to other mobile electronic devices by using an audio plug inserted into the audio socket in the mobile electronic device. In step 1021, in a preferred embodiment of the present invention, Fourier transform is used to accomplish the demodulation. In practice, fast Fourier Transform may be adopted for the process.

Accordingly, the audio signal may be transmitted through a wireless transceiver module in the mobile electronic device to other wireless devices. In this embodiment the audio signal is modulated and sent to the wireless transceiver module which has built-in application protocols, such as Bluetooth or near field communication.

In step 103, by calculating the skin conductance slope, the excitement degree is obtained. The sampling frequency is 2 Hz. The first order difference is used as the estimation SCRₛₗₒₚₑ of the skin conductance slope. After step 103, an additional step of playing multimedia files in response to the skin conductance slope 104 is added to soothe user's feeling.

Even though skin conductance response (SCR) has been studied in the past, none of the studies are directly or inherently related to the measurement of excitement degree, let alone any statistical analysis on multiple subjects to measure the influence of stimulation materials, such as films and music, in each of the designated time frame of the stimulation materials.

In conclusion, SCRₛₗₒₚₑ is able to reflect excitement or calmness degree and has better temporal resolution and effectiveness, which is the primary innovation and value of this application.

From the above description, it concludes that the present invention has the following advantages:

1. The Wheatstone bridge is adopted for measurement of skin conductance, and a transistor is used to amplify the conductance result. Modulating the conductance result into an audio signal helps the calculation of the skin conductance slope at a sampling frequency of 2 Hz to accurately measure the skin conductance variation and hence the emotion variation.

2. By transforming the skin conductance into an audio signal and by the coupling of an audio plug with an audio socket, the audio signal is easily and readily applied to a mobile electronic device such that the user may detect the excitement degree anytime and anywhere.

3. The system and method of detecting excitement degree targeted by the invention exploit the activation of sweat gland for skin conductance measurement. By measuring the rise of skin conductance, and calculating the slope thereof, the excitement degree is readily available for reference. With the combination of a mobile electronic device, the excitement information resulted from SCR modulation enables the mobile electronic device to provide more interesting functions and user interfaces. And the mobile electronic device so selected is able to display a suitable multimedia file, e.g. music, film, or picture to cope with the listener's emotion. As a result, the present invention provides an emotion-based multimedia retrieval technique.

It is to be noted that although the preferred embodiment of the present invention has been described, other modifications, alterations or minor change to the structure should still be within the scope defined in the claims. As those skilled in the art will readily observe that numerous modifications and alterations of the device and method may be made while retaining the teachings of the invention.

What is claimed is:

1. An emotion excitement detection system consisting essentially of:
a detection unit for engagement with human skin;
an amplifier unit connected to the detection unit to amplify signal detected by the detection unit;
a first control unit connected to the amplifier unit for measurement of skin conductance after the detection unit engages with the human skin and transmit result of the measurement; and
a processing unit computing a skin conductance slope in response to the skin conductance and excitement degree resulted from the skin conductance slope.

2. The system as claimed in claim 1, wherein the first control unit modulates the skin conductance into an audio signal for transmission.

3. The system as claimed in claim 2 further comprising a mobile electronic device to display the excitement degree.

4. The system as claimed in claim 3, wherein the mobile electronic device has a second control unit and the processing unit, the second control unit is to receive the audio signal and demodulate the audio signal into a skin conductance signal, the processing unit produces the skin conductance slope in response to the skin conductance signal.

5. The system as claimed in claim 3, wherein the mobile electronic device further includes a wireless transceiver module for transmitting the audio signal.

6. The system as claimed in claim 1 further comprising a play unit to play multimedia files in response to the skin conductance slope.

7. An emotion excitement method comprising the steps of:
measuring skin conductance;
amplifying the skin conductance; and
computing a skin conductance slope based on the skin conductance.

8. The method as claimed in claim 7 further comprising steps of modulating the skin conductance into an audio signal; and demodulating the audio signal into a skin conductance signal after the amplifying step.

9. The method as claimed in claim 8, wherein in the skin conductance transforming step, a pulse width modulation technique is adopted for the modulation.

10. The method as claimed in claim 8, wherein in the audio signal transforming step, the audio signal is processed to obtain the frequency of the audio signal and then the audio signal is demodulated to obtain the skin conductance signal.

11. The method as claimed in claim 7 further comprising a step of transmitting the audio signal through a wireless transceiver module to a wireless device.

12. The method as claimed in claim 7 further comprising a step of playing a suitable multimedia file format in response to the skin conductance slope.
